(19) United States
(12) Patent Application Publication
(10) Pub. No.: US 2008/0033215 A1
(43) Pub. Date: Feb. 7, 2008

(54) Insulated perfluoropolyether alkyl alcohols

(76) Inventors: Chadron Mark Friesen, British Columbia (CA); Kevin Anthony Hay, British Columbia (CA); Jon Lee Howell, Bear, DE (US); Daryl Allan Nyvall, British Columbia (CA)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/904,896

(22) Filed: Sep. 28, 2007

Related U.S. Application Data
(62) Division of application No. 11/156,348, filed on Jun. 17, 2005.

(51) Int. Cl. C07C 41/14

(52) U.S. Cl. 568/677

(57) ABSTRACT

Perfluoropolyether alkyl alcohol comprising a perfluoropolyether segment and one or more alcohol segments wherein the alcohol segment has a general formula, —CH₂(C_{q}H_{2q})OH, wherein C_{q}H_{2q} represents a divalent linear or branched alkyl radical where q is an integer from 1 to about 10, such that the hydroxy group is insulated from fluorine atoms, are disclosed. Also disclosed herein are processes to produce these perfluoropolyether alkyl alcohols by reaction of perfluoropolyether primary or secondary bromides or iodides either an alkene or alkenol followed by further reaction to produce the alcohol.

### BACKGROUND OF THE INVENTION

The synthesis of perfluoroalkyl alkanols, such as perfluoroalkylethyl alcohols and perfluoroalkylpropyl alcohols, from telomer iodides (F(CF₂CF₂)ₙI, prepared from tetrafluoroethylene and pentafluoroethyl iodide) has been described by Beck in U.S. Pat. No. 5,097,090 (perfluoroalkylethyl alcohols) and by Brace in J. Fluorine Chem. 1982, 20, 313 (perfluoroalkylpropyl alcohols). Derivatives of the perfluoroalkylethyl alcohols have found wide use as low surface tension products conferring soil, oil, and water repellency to a wide range of substrates.

Le Bleu, et al., in U.S. Pat. No. 3,293,306, describe perfluorinated ether alcohols including the structure XCF₂CF₂O(CFXCF₂O)ₙCFXCH₂OH wherein X is F or CF₃ and n is an integer from 1 to 50. The perfluorinated ether alcohols are prepared by reduction of the acid fluorides that result from the polymerization of, inter alia, hexafluoropropylene oxide. The acid fluorides have the structure CF₃CF₂CF₂O[CF(CF₃)CF₂O]ₙCF(CF₃)COF.

It is desirable to have a new family of fluorinated alcohols based on perfluoropolyethers corresponding to the versatile perfluoroalkyl alkanols. The present invention provides such perfluoropolyether alcohols and processes therefor.

### SUMMARY OF THE INVENTION

The present invention provides a perfluoropolyether alkyl alcohol comprising a perfluoropolyether segment and one or more alcohol segments, wherein the alcohol segment has a general formula, —CH₂(C_{q}H_{2q})OH, wherein C_{q}H_{2q} represents a divalent linear or branched alkyl radical where q is an integer from 1 to about 10. These alcohols are termed “insulated” alcohols, where “insulated” is defined hereinbelow.

Also provided in this invention is a first process to prepare a perfluoropolyether alkyl alcohol comprising a first step which comprises contacting a perfluoropolyether primary or secondary bromide or iodide with a terminal alkene followed by followed by a second step which comprises hydrolysis and sulfite treatment to produce the perfluoropolyether alkyl alcohol. There is also provided a second process to prepare a perfluoropolyether alkyl alcohol comprising a first step which comprises contacting a perfluoropolyether primary or secondary bromide or iodide with a terminally unsaturated alkenol followed by a second step which comprises treating with a metal hydride reagent to produce the perfluoropolyether alkyl alcohol.

Hypothetical fluoroalcohols of the structure 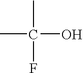
are unstable due to elimination of HF. For a fluoroalcohol to be stable, one or more fluorine-free carbon atoms must separate the alcohol group from adjacent fluorinated carbon atoms. Thus, a compound having the general formula R_{f}(CH₂)ₙOH where R_{f} is a perfluoroalkyl or perfluoropolyether group, is stable when n is greater than or equal to 1. Alcohols stabilized by such hydrocarbon groups separating the R_{f} group from the alcohol are herein termed “insulated” alcohols, because the hydroxy group is insulated from the fluorine atoms to prevent elimination of HF. Although compounds where n=1 are known (see, Le Bleu, et al., in U.S. Pat. No. 3,293,306, supra), compounds where n is greater than 1 have been heretofore unknown. As the value of n increases the electron withdrawing effect of the fluorocarbon segment is reduced, enhancing the insulating effect. Additionally, in compounds where n is greater than 1, the acidity of the alcohol is reduced and the stability of the corresponding esters with alkanoic acids is improved relative to compounds where n=1.

### DETAILED DESCRIPTION

Tradenames herein are shown in upper case.

The compositions of the present invention are perfluoropolyether alkyl insulated alcohols comprising a perfluoropolyether segment and one or more alcohol segments, wherein the alcohol segment has a formula —CH₂(C_{q}H_{2q})OH, wherein C_{q}H_{2q} represents a divalent linear or branched alkyl radical where q is an integer from 1 to about 10. The perfluoropolyether alkyl alcohol may be a mono-alcohol, a diol, or a polyol. By “mono-alcohol” it is meant herein that the perfluoropolyether alkyl alcohol consists of one segment having the formula —CH₂(C_{q}H_{2q})OH. By “diol” it is meant herein that the perfluoropolyether alkyl alcohol consists of two segments having the formula —CH₂(C_{q}H_{2q})OH. By “polyol” it is meant herein that the perfluoropolyether alkyl alcohol consists of three or more segments having the formula —CH₂(C_{q}H_{2q})OH.

Compositions of this invention wherein the perfluoropolyether alkyl alcohol is a mono-alcohol may have a general formula, R_{f}—CH₂(C_{q}H_{2q})OH, wherein R_{f} comprises a monovalent perfluoropolyether segment. Compositions of this invention wherein the perfluoropolyether alkyl alcohol is a diol may have a general formula, HO(C_{q}H_{2q})CH₂—R′_{f}—CH₂(C_{q}H_{2q})OH, wherein R′_{f} comprises a divalent perfluoropolyether segment.

Preferably, a composition of the present invention has the formula of:
F(C₃F₆O)_{z}CF(CF₃)CF₂CH₂(C_{q}H_{2q})OH;
F(C₃F₆O)_{z}CF(CF₃)CH₂(C_{q}H_{2q})OH;
HO(C_{q}H_{2q})CH₂(CF₂)ₐ(CF₂O)ₘ(C₂F₄O)ⱼ(CF₂)ₐCH₂(C_{q}H_{2q})OH;
F(C₃F₆O)_{y}(CF₂O)ₘCF₂CH₂(C_{q}H_{2q})OH;
F(C₃F₆O)_{y}(C₂F₄O)ⱼ(CF₂O)ₘCF₂CH₂(C_{q}H_{2q})OH;
HO(C_{q}H_{2q})CH₂CF₂CF(CF₃)O(C₃F₆O)ₚR_{f}²O(C₃F₆O)ₖCF(CF₃)—CF₂CH₂(C_{q}H_{2q})OH;
HO(C_{q}H_{2q})CH₂CF₂CF(CF₃)O(C₃F₆O)ₚR_{f}²O(C₃F₆O)ₖ—CF(CF₃)CH₂(C_{q}H_{2q})OH;
HO(C_{q}H_{2q})CH₂CF₂CF₂O(C₃F₆O)ₓCF(CF₃)CF₂CH₂(C_{q}H_{2q})OH;
HO(C_{q}H_{2q})CH₂CF₂CF₂O(C₃F₆O)ₓCF(CF₃)CH₂(C_{q}H_{2q})OH;
R_{f}¹O(CF(R_{f}³)CF₂O)_{w}CF(R_{f}³)CF₂CH₂(C_{q}H_{2q})OH;
R_{f}¹O(CF(R_{f}³)CF₂O)_{w}CF(R_{f}³)CH₂(C_{q}H_{2q})OH;
R_{f}¹O(CF(CH₂(C_{q}H_{2q})OH)CF₂O)ᵤ(C₃F₆O)_{z}CF₂CF₃;
HO(C_{q}H_{2q})CH₂CF₂CF₂CF₂O(CF(R_{f}³)CF₂O)_{w}CF(R_{f}³)CF₂CH₂(C_{q}H_{2q})OH;
HO(C_{q}H_{2q})CH₂CF₂CF₂CF₂O(CF(R_{f}³)CF₂O)_{w}CF(R_{f}³)CH₂(C_{q}H_{2q})OH;
HO(C_{q}H_{2q})CH₂CF₂CF₂CF₂O(CF(R_{f}³)CF₂O)_{w}CF₂CF₃; or
(R_{f}¹)(R_{f}¹)CFO(C₃F₆O)ₓCF(CF₃)CF₂CH₂(C_{q}H_{2q})OH;
wherein

u is a number from 1 to about 100;

w is a number from 2 to about 100;

x is a number from 2 to about 100;

y is a number from 2 to about 100;

z is a number from about 3 to about 100, preferably 3 to about 50, more preferably about 4 to about 25, and even more preferably about 4 to about 15;

p is a number from 2 to about 50;

j is a number from 2 to about 50;

k is a number from 2 to about 50;

m is a number from 2 to about 50;

a is 1 or 2;

each R_{f}¹ can be the same or different and is independently a monovalent C₁ to C₂₀ branched or linear fluoroalkane;

R_{f}² can be the same or different and is independently a divalent C₁ to C₂₀ branched or linear fluoroalkyl group;

R_{f}³ can be the same or different and is independently CF₃ or CH₂C_{q}H_{2q}OH;

C₃F₆O is linear or branched; and

C_{q}H_{2q} is as defined above.

More preferably, the perfluoropolyether alkyl alcohol composition of this invention is a mono-alcohol having a general formula, R_{f}⁴—CH₂(C_{q}H_{2q})OH, wherein R_{f}⁴ comprises a perfluoropolyether segment and C_{q}H_{2q} is defined above. Still more preferably, the perfluoropolyether alkyl alcohol composition of this invention has the formula of F(C₃F₆O)_{z}CF(CF₃)CF₂CH₂(C_{q}H_{2q})OH, wherein z and C_{q}H_{2q} are the same as defined above.

The perfluoropolyether alkyl insulated alcohols of the present invention can be produced by any means known to one skilled in the art. Conveniently and preferably, the compositions are produced by a process disclosed herein starting from perfluoropolyether primary or secondary bromides and iodides.

Perfluoropolyether primary and secondary bromides and iodides may be prepared by any means known to one skilled in the art, such as the processes described by Howell, et al., in U.S. Pat. No. 6,653,511, the teachings of which are incorporated herein by reference.

In a first process embodiment, the present invention provides a process for the preparation of perfluoropolyether alkyl insulated alcohols comprising (a) contacting a perfluoropolyether primary or secondary bromide or iodide with an alkene to produce a perfluoropolyether alkyl bromide or iodide; and (b) hydrolyzing the product of step (a) by sequentially contacting the perfluoropolyether alkyl bromide or iodide with (1) oleum and (2) a sulfite, such as sodium sulfite, to produce a perfluoropolyether alkyl insulated alcohol.

In this embodiment, in a first step, a perfluoropolyether primary or secondary bromide or iodide is contacted with a stoichiometric excess of a terminal alkene, for example, but not limited to ethylene or propene. The reaction may be accelerated by increased temperature. The reaction is typically performed at a temperature from about 50° C. to about 300° C., preferably from about 150° C. to about 250° C. and more preferably about 200° C. The reaction may be accelerated by any increased pressure. The alkene is inserted between the terminal CF₂ group and the halide atom. In the ethylene and propene examples described, when the halide is iodide, the terminal groups are —CF₂CH₂CH₂I and —CF₂CH₂CHICH₃, respectively. The perfluoropolyether alkyl bromides and iodides are intermediates for the preparation of the perfluoropolyether alkyl insulated alcohols.

In this first embodiment, in a second step, a perfluoropolyether alkyl bromide or iodide undergoes acid hydrolysis. In this step, the perfluoropolyether alkyl bromide or iodide prepared in the first step is contacted with oleum, followed by addition of a sulfite salt. A solution of an alkali metal sulfite is conveniently used, especially an aqueous solution of sodium sulfite. The acid hydrolysis step is analogous to the conversion of perfluoroalkyl iodides as described by Beck in U.S. Pat. No. 5,097,090, supra.

In a second process embodiment, the present invention provide a process for the preparation of a perfluoropolyether alkyl insulated alcohol comprising (a) contacting a perfluoropolyether primary or secondary bromide or iodide with a terminally unsaturated alkenol in the presence of a radical initiator or a transition metal catalyst to produce a perfluoropolyether bromide or iodide alkanol; and (b) contacting the product of step (a) with a metal hydride reagent to produce a perfluoropolyether alkyl insulated alcohol.

In this embodiment, in a first step, a perfluoropolyether primary or secondary bromide or iodide is contacted with a stoichiometric excess of a terminally unsaturated alkenol, for example, but not limited to, allyl alcohol. Contact of perfluoropolyether primary or secondary bromide or iodide with the alkenol is performed in the presence of a radical initiator or a transition metal catalyst. The radical initiator is preferably an azo radical initiator, such as 2,2′-azobis(isobutyronitrile) (commonly referred to as AIBN). The transition metal catalyst is preferably copper. Conversion is enhanced by increasing the amount of excess alkenol. The reaction may be performed at an elevated temperature, for example, to increase rate and/or to provide an appropriate decomposition rate of the initiator. Preferably, the reaction is performed under an inert atmosphere, such as nitrogen.

In this second embodiment, in a second step, the product of the first step, that is, a perfluoropolyether bromide or iodide alkanol, such as, for example, perfluoro-2-iodo-3-alkanol, when a perfluoropolyether iodide and allyl alcohol are used, is reduced to the desired perfluoropolyether alkyl insulated alcohol. This reduction step is performed using a metal hydride reagent, such as tributyl tin hydride. The reduction step is preferably performed at an elevated temperature, such as above about 50° C., for example, at about 80° C. An appropriate inert solvent, such as trifluorotoluene is also used.

The perfluoropolyether alkyl insulated alcohols can be used for or as:

Lubricants for the magnetic computer hard-drive industry,

Reactive intermediates in polymerization reactions,

Fluorous Biphase Catalysis intermediates,

Fluorous separations technologies,

Fluorous proteomics,

Formation of new ionic liquids,

Preparation of Lubricant Additives with Anticorrosion Properties,

Surfactants, for example, in oxygen line cleaning,

Formation of stain resistant and oil and water repellency coatings for various fabric articles.

For alternative uses, see Chapters 8 and 14 of Organofluorine Chemistry—Principles and Commercial Applications; Banks, R. E., Smart, B. E., Tatlow, J. C., Eds.; Plenum Press: New York and London, 1994.

### Materials and Test Methods

KRYTOX Iodide, F(CF(CF₃)CF₂O)_{z}CF(CF₃)CF₂I, where z has an average value of about 8, is produced by the methods described in U.S. Pat. No. 6,653,511, incorporated herein by reference.

### EXAMPLES

¹H NMR data for the Examples is provided in the Table below.

### Example 1

### Preparation of F[CF(CF₃)CF₂O]_{z}CF(CF₃)CF₂CH₂CH₂I

### “Compound 1”

A high-pressure 1800-psig (12.5 MPa), 304 ss, 0.25 inch (0.64 cm) female National Pipe Thread (NPT) single ended 150-mL HOKE Cylinder (Peacock, Edmonton, AB) fitted with a 400 psig (2.86 MPa) ss-gauge, SWAGELOK 0.25 inch (0.64 cm) male NPT×female NPT street-tee, and a 316 ss, straight 0.25 inch (0.64 cm) male NPT×SWAGELOK needle valve was charged with polyhexafluoropropylene oxide primary iodide (87 g, 64.7 mmol) and degassed under vacuum at 25° C. Ethylene (3.37 g, 129.6 mmol) was then transferred to the same cylinder under static vacuum at −196° C. and then slowly warmed to 25° C., when a pressure of 238 psig, 1.74 MPa was measured). The contents were heated to 220-250° C. for 24 h. The pressure reached as high as 300 psig (2.17 MPa) during the reaction and around 150 psig (1.14 MPa) when cooled after 24 h to 25° C. After the reaction was completed, the excess ethylene was vacuum-stripped to give a light tan oil (78.38 g, 88% yield). The spectroscopic (nuclear magnetic resonance and mass spectrometry) evidence was consistent with the characteristics of the desired compound.

Example 1 demonstrates the insertion of an ethylene group into the —CF₂—I terminal group.

### Example 2

### Preparation of F[CF(CF₃)CF₂O]_{z}CF(CF₃)CF₂CH₂CH₂OH

### “Compound 2”

To a 250-mL three-necked flask was added 50 mL of 20% oleum. The flask was fitted with a reflux condenser. The reaction apparatus was flushed with nitrogen. Compound 1 (10 g, prepared as above was added slowly to the oleum using a separatory funnel (a syringe was an alternative) over the period of an hour; the reaction darkened to a black color. The temperature was maintained at about 90° C. The black reaction mixture was then hydrolyzed by pouring the acid mixture into 125 mL of 1.5% sodium sulfite solution (Na₂SO₃(aq), 15.161 g in 1 L of water) in an ice bath over a 10-minute period. The color of the reaction mass became a clear light yellow. Ethanol (200 mL) was added after the aqueous sulfite solution and the alcoholic mixture was refluxed at 100° C. and the end of the hydrolysis reaction was detected by GC/MS. The reaction took up to 10 or more hours. As the alcohol (Compound 2) forms it separates from the acid solution, forming a two-phase system. Following separation, the yellowish oil was filtered using diatomaceous earth to provide Compound 2, (6.10 g, 76.2% yield). The spectroscopic (nuclear magnetic resonance and mass spectrometry) evidence was consistent with the characteristics of the desired compound.

Example 2 demonstrates the conversion of the —CF₂CH₂CH₂I terminal group, prepared in Example 1, into the —CF₂CH₂CH₂OH terminal alcohol.

### Example 3

### Preparation of F[CF(CF₃)CF₂O]ₙCF(CF₃)CF₂CH₂CHICH₂OH

### “Compound 3”, using AIBN

Poly-hexafluoropropylene primary iodide (30.4 g, 22.6 mmol), allyl alcohol (1.75 g, 30.2 mmol), and 2,2′-azobis(isobutyronitrile) (AIBN, 50 mg, 0.43 mmol) were heated under positive nitrogen pressure for 86 h at 90° C. in a 250-mL round-bottomed flask fitted with a reflux condenser, thermometer, and magnetic stirrer. The reaction was monitored every 24 h by GC/MS. If the reaction had not progress any noticeable amount, additional AIBN (50 mg) was added. When the reaction was complete as evidenced by the GC/MS, the product was washed in a separatory funnel with three 20-mL portions of acetone. The product (Compound 3) was a brown oil (27.1 g, 85% yield).

The spectroscopic (nuclear magnetic resonance and mass spectrometry) evidence was consistent with the characteristics of the desired compound.

Example 3 demonstrates the conversion of the —CF₂—I terminal group into the —CF₂CH₂CHICH₂OH iodo-alcohol using AIBN.

### Example 4

### Preparation of F[CF(CF₃)CF₂O]ₙCF(CF₃)CF₂CH₂CHICH₂OH

### “Compound 4”, Using Cu

Poly-hexafluoropropylene primary iodide (5.0 g, 3.21 mmol) and allyl alcohol (5.0 mL, 73.5 mmol) were heated to 95° C. under positive nitrogen pressure and then Cu (0.104 g, 0.24 mmol, previously washed with 1.0M HNO₃) was added and allowed to react for 72 h. The reaction was carried out in a 250-mL round-bottomed flask adapted with a reflux condenser, thermometer, and magnetic stirrer. Upon completion of the reaction as monitored by GC/MS, the product was filtered through diatomaceous earth and vacuum stripped of any residual allyl alcohol. The product was identical in all aspects to Compound 3, which is a clear oil (100% conversion, 87% yield).

Example 4 demonstrates the conversion of the —CF₂I terminal group into the —CF₂CH₂CHICH₂OH iodo-alcohol using allyl alcohol and Cu catalyst, an alternative synthesis to Example 3.

### Example 5

### Preparation of F[CF(CF₃)CF₂O]ₙCF(CF₃)CF₂CH₂CH₂CH₂OH

### “Compound 5”

Compound 4 (27.1 g, prepared according to Example 4), was placed in a round-bottomed flask with 50 mL of trifluorotoluene and AIBN (400 mg). The contents were heated under positive nitrogen pressure at 80° C. in a 250-mL round-bottomed flask fitted with a reflux condenser, thermometer, and magnetic stirrer. Next, tributyl tin hydride, (Bu)₃SnH (3 mL, 11.4 mmol) was added dropwise over 5 min. Heating of the reaction at 80° C. was continued for 5 h. After the reaction, the product was washed in a separatory funnel with three 20-mL portions of acetone. The product (Compound 5) was a brown oil (15.4 g, 62.4% yield).

The spectroscopic (nuclear magnetic resonance and mass spectrometery) evidence was consistent with the characteristics of the desired compound.

Example 5 demonstrates the reduction of the terminal —CF₂CH₂CHICH₂OH iodo-alcohol to the —CF₂CH₂CH₂CH₂OH terminal alcohol using tributyl tin hydride.

TABLE
¹H NMR DATA

| | Example | | | | |
|---|---|---|---|---|---|
| | 1 | 2 | 3 | 4 | 5 |
| ¹H NMR (400 MHz, | | | | | |
| CDCl₃) δ | | | | | |
| R_{f}—CH₂—CH₂ I | 2.7 (m) | | | | |
| R_{f}—CH₂—CH₂ I | 3.3 (t) | | | | |
| R_{f}CH₂—CH₂ OH | | 3.7 (t) | | | |
| R_{f}CH₂—CH₂ OH | | 2.2 (m) | | | |
| R_{f}CH₂—CHI—CH₂ OH | | | 4.4 (m) | 4.4 (m) | |
| R_{f}CH₂—CHI—CH₂ OH | | | 2.9 (m) | 2.9 (m) | |
| R_{f}CH₂—CHI—CH₂ OH | | | 3.8 (m) | 3.8 (m) | |
| R_{f}CH₂—CH₂—CH₂ OH | | | | | 3.8 (t) |
| R_{f}CH₂—CH₂—CH₂OH | | | | | 1.9 (m) |
| R_{f}CH₂—CH₂—CH₂OH | | | | | 2.3 (m) |

1-19. (canceled)

20. A process for the preparation of a perfluoropolyether alkyl insulated alcohol comprising (a) contacting a perfluoropolyether primary or secondary bromide or iodide with a terminally unsaturated alkenol in the presence of a radical initiator or a transition metal catalyst to produce a perfluoropolyether bromide or iodide alkanol; and (b) contacting the product of step (a) with a metal hydride reagent to produce a perfluoropolyether alkyl insulated alcohol.

21. The process of claim 20 wherein the alkenol is allyl alcohol.

22. The process of claim 20 wherein step (a) is performed in the presence of a radical initiator.

23. The process of claim 22 wherein the radical initiator is 2,2′-azobis(isobutyronitrile).

24. The process of claim 20 wherein step (a) is performed in the presence of a transition metal catalyst.

25. The process of claim 24 wherein the catalyst is copper.

26. The process of claim 20 wherein step (a) is performed in an inert atmosphere.

27. The process of claim 20 wherein the metal hydride reagent is tributyl tin hydride.
